(19) United States
(12) Patent Application Publication
(10) Pub. No.: US 2004/0127859 A1
(43) Pub. Date: Jul. 1, 2004

(54) Anti-reflux syringe

(76) Inventor: Michael Terrance Ward, Libertyville, IL (US)

Correspondence Address: James P. Hahrath, Boundas, Skarzynski, Walsh & Black, LLC, 200 East Randolph Drive, 72nd Floor, Chicago, IL, 60601, US

(21) Appl. No.: 10/329,937

(22) Filed: Dec. 26, 2002

(51) Int. Cl. A61M 5/315

(52) U.S. Cl. 604/222

(57) ABSTRACT

An anti-reflux syringe includes a syringe barrel defining a chamber for retaining fluid that has an open proximal end, a distal portion, and a barrel extension tip having a passageway therethrough in fluid communication with the chamber. A plunger stem operative of a piston and elastomeric stopper assembly controls the flow of fluid within the chamber. The piston includes a distal piston extension member located distal of the stopper having a male retention body on its outer periphery capable of being retained within a cooperative female retention element in the interior surface of the barrel extension tip. This retention at a point distal of the stopper establishes a predetermined limit to the distal extension of the elastomeric stopper to avoid an undue compression of the same that upon release or withdrawal of the stopper may cause reflux. Control of the stopper may also be established at a point proximal of the stopper either by a handle of the piston stem coming into lock contact with a capturing detent of a proximal extension of the flange of the open proximal end of the syringe barrel or by one or more detents on a proximal portion of the internal surface of the syringe barrel allowing distal passage of one or more fins on the exterior surface of the plunger stem while preventing or hindering retraction thereof.

### BACKGROUND OF THE INVENTION

1. Field of the Invention

The present invention relates generally to syringe assemblies, especially to flush syringes for use in I.V. flush procedures, and more particularly to syringe assemblies having structure to limit and control compression of an elastomeric stopper of a syringe barrel piston to avoid reflux during syringe barrel fluid dispersion.

2. Description of the Related Art Including Information Disclosed Under 37 CFR 1.97-1.99.

Modern medical practice commonly employs intravenous (I.V.) solutions to administer medications to patients. Many patients, in accordance with their medical treatments, have an I.V. catheter connected to a vein ready for use in various procedures or in fluid communication with an I.V. system for infusing liquids and medications.

A wide variety of I.V. sets exist of various complexity having I.V. ports that are in fluid communication with a catheter and allow access for the purpose of injecting medication into the patient, and for use in periodic flushing techniques and protocols to maintain catheter potency and integrity.

The most common I.V. ports are covered by pierceable septums or pre-slit septums preferably made of rubber or another elastomeric material that permits insertion of a sharp needle cannula in order to infuse fluids into or to withdraw fluids from the catheter. Upon withdrawal of the needle cannula the septum closes upon itself to again form a seals. However use of needle cannula may present a disadvantage if the septum can be “cored” by the needle cannula so that a core of material is removed from the septum creating a possible passage. Also, repeated piercing of the septum by a needle cannula may result in a permanent opening being form through the septum thereby defeating its purpose.

Ports having pre-slit septums are used with blunt cannula. Typically, the blunt cannula is attached to a syringe and the syringe is moved to place a gentle pressure on the pre-slit septum which is forced open by the blunt cannula to establish fluid communication. A slit septum, however, may lose elasticity or weaken such that it may not be rigid enough to withstand back pressure from the fluid line when the cannula is removed. In such an event, the integrity of the slit septum as a site valve is jeopardized which could result in fluid leakage from the site valve with the attended undesirable risk of infection and/or disease transmission.

Catheters are flushed using syringe assemblies filled with various fluids. For example, catheter lines may be periodically flushed with saline flush solution and/or heparin lock flush solution depending on the protocol. Among other things, flushing saline solution removes blood from the catheter and heparin, an anticoagulant, helps prevent the formation of future blood clots.

The size of the syringe used to flush I.V. lines varies by various factors including the size and length of the catheter. Typically syringes of 1 ml, 3 ml, 5 ml and 10 ml volume are used. A commercially available 1 ml syringe may have a barrel inside diameter of approximately 6.6 mm (0.26 inch) a 3 ml syringe may have a barrel inside diameter of approximately 8.6 mm (0.34) inch while a 10 ml syringe may have a barrel inside diameter of approximately 14.5 mm (0.57 inch). Unfortunately, during the flushing of vascular access devices, different nominal size syringes generate substantially different pressures in the solution being injected by the same force being applied to the syringe plunger rod. For example, a ten-pound force on the plunger rod may cause a 10 ml syringe to generate 40 psi of liquid pressure while a 3 ml syringe generates 110 psi and a 1 ml syringe generates 190 psi.

Control of pressure is very important during flush procedures for detection of resistance to flow or catheter occlusion, and to avoid over-pressure because of the danger of dislodging a clot or rupturing the catheter. It is important to maintain a positive pressure during the flush procedure. The elastomeric stopper of a plunger rod of a flush syringe can be compressed when it contacts the distal end of the chamber in the syringe barrel. If the user compresses the stopper and then relieves the pressure on the plunger rod flange, the stopper will expand back to its normal size drawing liquid from the catheter into the syringe barrel. This is undesirable since this event can cause blood to undesirably enter the catheter at the catheter distal end where it can clot and seal the catheter.

Thus, in order to prevent blood reflux into the catheter the user is encouraged to maintain a positive pressure in the line during the flush procedure.

Although a wide variety of catheters and I.V. ports can be adequately flushed using currently available syringe assemblies, there is still a need for simple, easy to use, and easy-to-manufacture syringe assemblies which minimize the potential for reflux accidentally drawing blood in the catheter during the flush procedure.

In U.S. Pat. No. 6,361,524 to Odell et al. there is disclosed a syringe assembly addressing the foregoing need. The syringe assembly of this patent comprising a syringe barrel having an elongated body defining a chamber for retaining fluid, an open proximal end, a distal end, and a frusto-conically shaped tip extending from the distal end and having a passageway therethrough in fluid communication with the chamber. A stopper in fluid tight engagement inside the syringe barrel is operative with an elongated plunger rod that extends proximally from the stopper through the open end of the syringe barrel. In one embodiment of the Odell et al. syringe assembly, a flange at the proximal end of the plunger rod is shaped and positioned to limit the distal motion of the plunger rod in the barrel by contacting the proximal end of the barrel. The contact of the plunger flange upon the open proximal end of the barrel limits the stroke of the plunger rod and stopper to prevent the stopper from being excessively compressed against the distal chamber wall of the syringe barrel. Hence, by controlling the distal face of the stopper to be for example, at least partially spaced from the distal chamber wall of the syringe barrel, the syringe assembly is designed to avoid substantial compression of the stopper so as to prevent or substantially minimize undesirable reflux. In another embodiment of the Odell et al. syringe assembly, the diameter of the syringe barrel chamber is at least 13.5 mm (0.53 inches), the length of the syringe barrel chamber is no more than about 57 mm (2.25 inches), and the ratio of the inside diameter of the syringe barrel chamber to that of its distal tip passageway in fluid communication with the chamber is selected to produce substantially lower pressure in the flush solution injected through the passageway than such pressure in a conventional syringe that contain substantially similar volume of flush solution.

### SUMMARY OF THE INVENTION

According to the present invention there is provided a syringe device comprising (a) a syringe barrel having an elongated body with an external surface and an internal surface defining a chamber for retaining fluid, an open proximal end, a distal portion, and a barrel extension tip extending from the distal portion, the barrel extension tip having a tip passageway therethrough in fluid communication with the chamber; (b) a piston disposed in the syringe barrel for controlling the flow of fluid within the elongated body, the piston having a distal piston extension member capable of engaging the barrel extension tip; (c) a plunger stem having a distal end operatively connected to the piston and a proximal end terminating into a handle, the handle being disposed to extend proximally from the open proximal end of the syringe barrel, the plunger stem being movable with the piston; and (d) a stopper integral with or releaseably mounted upon the piston in fluid tight communication inside the chamber, the stopper having a bore at a distal portion thereof through which the distal piston extension member extends.

In a preferred embodiment of the syringe device, the distal piston extension member further includes a retention body at a portion of its outer periphery and the barrel extension tip further includes a retention element at an inner surface thereof, the retention body of the distal piston extension member being dimension to cooperatively engage the retention element of the barrel extension tip at a position distal of the syringe piston stopper. This cooperative engagement limits the distal movement of the stopper and defines a predetermined maximum distal position thereof. Additionally, the cooperative engagement importantly forms an inhibiting barrier to proximal retraction of the stopper. The syringe device retention body of the distal piston extension member preferably comprises a male protrusion and the retention element of the barrel extension tip preferably comprises a female reception area. The cooperative engagement of the retention body of the distal piston extension member and the retention element of the barrel extension tip may be either a releasable interference fit or a one use snap lock fit.

Control of the maximum distal extension of the elastomeric stopper for discharge of a liquid from a syringe barrel may include structure proximal of the stopper for limiting the distal movement of the stopper. Such structure may include the open proximal end of the syringe barrel terminating into a flange that, when contacted by a piston stem handle, limits and predetermines the distal movement of the piston stopper at a point at which the stopper is flush with or nearly flush with the distal end of the syringe barrel chamber.

The syringe device of the present invention also advantageously provides for structure proximal of the piston stopper for inhibiting proximal retraction of the stopper from a distally extended position of the same or at its maximum distal extension. For example, the syringe device may include a syringe barrel flange having a proximally extending branch that terminates into an inwardly extending detent capable of capturing a piston stem handle in a manner which affirmatively inhibits its proximal retraction or release thereby preventing decompression of any compressed state of the stopper at its maximum distally extended position that can cause reflux a. Still further, the inhibiting proximal structure may include the internal surface of the elongated body defining the syringe barrel chamber having one or more inwardly extending detents and the plunger stem operating the piston stopper having one or more fins extending from its outer surface wherein the detent is capable of allowing distal passage of the fin while hindering or preventing proximal retraction of the fin. In this regard, the detent of the syringe barrel chamber may comprise an annular ring, preferably located at a proximal end portion thereof. Alternatively the detent may comprise a distally facing edge terminating into proximally sloped surface cooperative with a fin comprising a proximally facing edged terminating into a distally sloped surface.

The present invention advantageously provides for a distally operative predetermined control of the elastomeric stopper of the piston distal end of a plunger stem optionally supplemented by stopper retraction inhibiting structure proximal of the stopper to avoid excessive compression of the stopper that, upon release of the plunger stem, can reflux draw blood into a catheter during the flush procedure.

Additional features and advantages of the present invention will become apparent to those skilled in the art from the following description and the accompanying figures illustrating preferred embodiments of the invention, the same being the present best mode for carrying out the invention.

### BRIEF DESCRIPTION OF THE DRAWINGS

FIG. 1 is a perspective view of a syringe device constructed in accordance with the teachings of the present invention wherein a syringe barrel assembly is vertically exploded from a piston plunger assembly.

FIG. 2 is a perspective view of the syringe device of FIG. 1 with its component parts vertically exploded from one another.

FIG. 3 is a perspective view of the distal end of the syringe device of FIG. 1 that illustrates a retention body of the distal piston extension member prior to its reception within a cooperative retention element of a syringe barrel extension tip.

FIG. 4 is a perspective view of the distal portion of the syringe device of FIG. 1 that illustrates the retention body of the distal piston extension member now seated within the cooperative retention element of the syringe barrel extension tip.

FIG. 5 is perspective view of an alternative embodiment of the of the syringe device of the present invention, that illustrates an alternative embodiment syringe barrel having an open proximal end terminating into a flange, the flange further including a proximally extending branch having an inwardly extending detent capable of capturing a handle of a piston plunger assembly.

FIG. 6 is a perspective view of the alternative embodiment syringe barrel of FIG. 5 wherein the plunger assembly is in a captured position.

FIG. 7 is a perspective view of an alternative embodiment of the syringe device of the present invention that illustrates an alternative embodiment syringe barrel having a plurality of inwardly extending detents suited to cooperatively engage a plurality of fins outwardly extending from the external surface of an alternative plunger stem.

FIG. 8 is a perspective view of the alternative embodiment syringe barrel of FIG. 9 wherein the inwardly extending detents have engaged the fins.

FIG. 9 is an end perspective view of an alternative embodiment of the inwardly extending detent of the syringe barrel wherein the detent now comprises an annular ring.

FIG. 10 is an end perspective view of another alternative embodiment of the inwardly extending detent of the syringe barrel wherein the detent now comprises a fragmented annular ring defining fin passage channels.

FIG. 11 is a packaged kit of a pre-filled syringe device of the present invention wherein the packaging consists of a sterile, tamper evident barrier.

### DESCRIPTION OF THE PREFERRED EMBODIMENTS

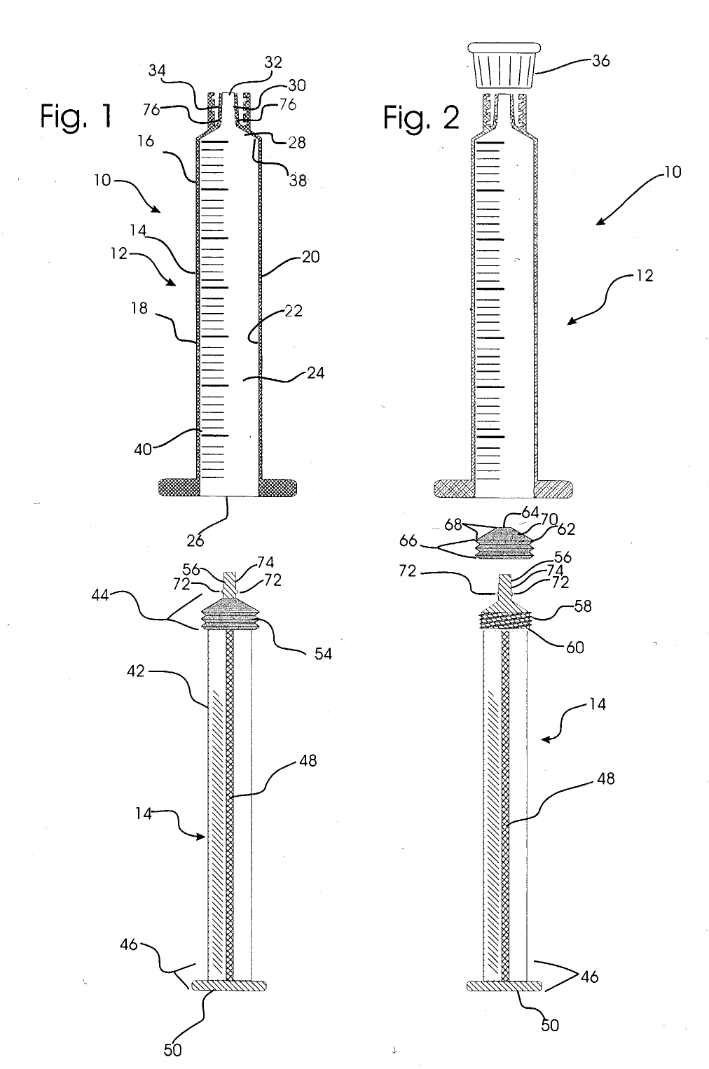

Referring now to FIG. 1, there is shown a syringe device 10 constructed in accordance with the teachings of the present invention. For purposes of the description of the present invention, the terms “distal” or “distal end” of an element is meant to refer to the portion or end of the element furthest from the person holding the syringe device of the present invention. The terms “proximal” or “proximal end” are used herein to refer to the portions or end of the element closest to the person holding the syringe device of the present invention. The syringe device 10 is generally of two-part construction having a syringe barrel assembly 12 and a piston plunger assembly *14. The syringe barrel assembly includes syringe barrel 16 having elongated body 18 with an external surface 20 and the internal surface 22 defining a chamber 24 for retaining an injectable fluid. The syringe barrel includes open proximal end 26 exposing the syringe barrel chamber 24, a distal portion 28 of the syringe barrel chamber 24 that terminates into a barrel extension tip 30 having a passageway 32 therethrough in fluid communication with the chamber 24. The barrel extension tip is preferably frusto-conically shaped. The external periphery 34 of the barrel extension tip 30 is preferably externally threaded to engage the internal recessed threads of a sealing luer cap 36 {see FIG. 2} for releasable connection to the barrel extension tip 30 thereby closing passageway 32. It is preferred that the luer cap 36 is formed of material selected from the group of thermoplastic material and elastomeric material with elastomeric material being preferred.

The length of the syringe barrel chamber may vary and is measured approximately between the interior facing surface 38 of the barrel extension tip 30 and the open proximal end 26 of the chamber 24. The chamber 24 is generally of constant diameter and of constant cross-section prior to the barrel extension tip thereof.

The syringe device of the present may have chamber 24 dimensioned of differing lengths and diameters to accommodate a varying desired volumes of injectable fluid. The syringe device 10 contains volume measuring indicia 40 on its syringe barrel elongated body 18. The volume measuring indicia in FIG. 1 for example shows 1.0 ml incremental volumes up to 12.0 ml. However, since the syringe device is preferably pre-filled with a selected injectable fluid, the indicia 40 may be limited to the exact amount of liquid in the syringe, for example, 12.0 ml., and may alternatively provide for printed or labeled instructions or a description of the syringe contents.

For many I.V. Flush procedures, it is preferred that the injectable fluid be selected from the group consisting of saline flush solution and heparin lock flush solution. These solutions are known in the art and readily available. An example of a saline flush solution is 0.9% Sodium Chloride USP. An example of a heparin lock flush solution is 0.9% Sodium Chloride with 100 USP units of Heparin Sodium per ml or 10 USP units of Heparin Sodium per ml.

The piston plunger assembly 14 of the syringe device 10 includes an elongated plunger stem 42, shown in FIG. 1 as being generally X-shaped in cross-section, that has a distal end 44 and a proximal end 46 defining a longitudinal axis 48. Plunger stem 42 terminates into a handle 50 at its proximal end 46 and into a piston 52 at its distal end 44. The plunger stem is preferably made of a rigid thermoplastic material. Piston 52 of the plunger stem 42 includes a stopper 54 that may be an integral or separate element. The stopper 54 is preferably made of an elastomeric material selected from the group of natural rubber, synthetic rubber, thermoplastic elastomers or combinations thereof. The stopper 54 is dimensioned for fluid-tight engagement inside the syringe barrel chamber 24.

In FIG. 1, stopper 54 is integral with piston 52. Piston 52 has a distal piston extension member 56 extending distally therefrom. In FIG. 2, the piston 52 includes distally directed threaded extensions 58 on its outer periphery 60 capable of engaging cooperative threaded recesses in an internal surface of a separate component piece stopper 62 that has a central axis bore 64 permitting the distal piston extension member 56 to extend through. The component piece stopper 62 has a proximal portion 66 containing internally exposed threaded recesses for mating with distally directed threaded extensions 58 of piston 52 and a distal portion 68 comprising a distal seating face 70 suited for setting within the interior facing surface 38 of barrel extension tip 30. Upon such a setting, the distal piston extension member 56 of piston 52 extends distally through bore 64 of the stopper 62 into tip passageway 32 of the barrel extension tip 30. The extension of the distal piston extension member 56 into tip passageway 32 may form a frictional fit serving to limit the compression of the elastomeric stopper 62 within the interior facing surface 38 of barrel extension tip 30. Such a frictional fit achieved by the distal piston extension member 56 serves to minimize reflux in the syringe device that does not contain additional mechanical syringe structure to inhibit proximal retraction of the piston stem, piston, and stopper assembly.

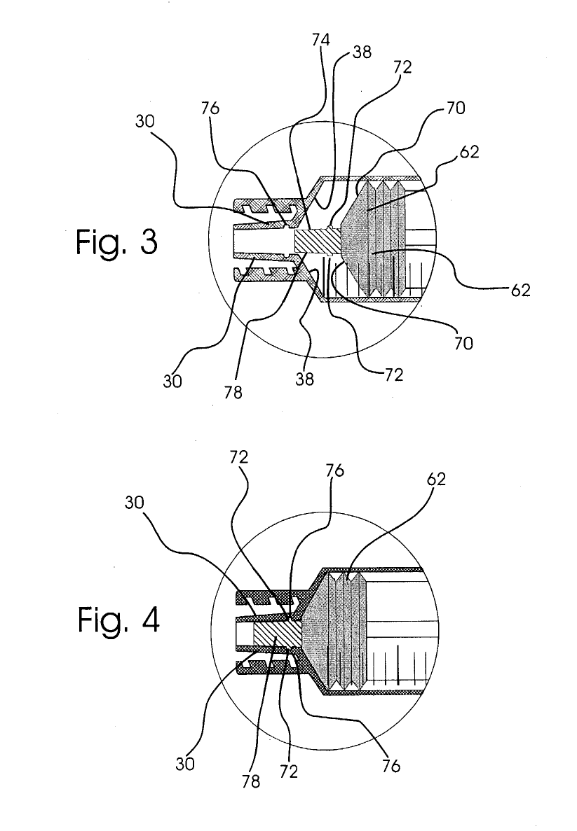

However, it is preferred that the distal piston extension member 56 include at least one retention body 72 at a portion of its outer periphery 74 and that the barrel extension tip 30 include a complementary retention element 76 at an adjacent portion of its interior facing surface 38 such that the retention body 72 may cooperatively engage the retention element 76 in either a retractable interference fit or a one use snap lock fit. This cooperative engagement is illustrated at FIGS. 3 and 4. FIG. 3 illustrates a pair of aligned retention bodies 72 at outer periphery 74 of the distal piston extension member 56 prior to their linear pathway reception within a pair of aligned cooperative retention elements 76 at the interior facing surface 38 of the barrel extension tip 30 of a syringe barrel extension tip 30. FIG. 4 illustrates the retention bodies 72 of the distal piston extension member 56 now seated within the cooperative retention elements 76 so as to position the distal piston extension member 56 in a predetermined seated engagement within the tip passageway 32 of the barrel extension tip 30. The engagement limits both the distal and proximal movement of the stopper distal seating face 70 of component piece elastomeric stopper 62 within the internal proximally facing surface 38 of barrel extension tip 30 to avoid undue compression of the stopper therein and its release that causes reflux. The cooperative engagement occurs at a position 78 distal of the stopper 54, 62 that limits the distal movement of the stopper, defines a predetermined maximum distal position of the stopper, and forms a barrier to proximal withdrawal of the stopper. The anti-reflux mechanical structure of the retention bodies 72 and retention elements 76 achieving the cooperative engagement is disposed distal of the stopper 54, 62, preferably at a point distally adjacent distal seating face 70 upon the predetermined maximum distal position of stopper 54, 62.

As illustrated at FIGS. 3 and 4, it is preferred that the retention body 72 of the distal piston extension member 56 comprise a male protrusion and the retention element 76 of the barrel extension tip comprise a female reception area so as to form complementary male and female mating structure to achieve the foregoing cooperative engagement. Such male and female mating structure may be of various configurations or of complimentary irregular surfaces capable of providing a distal seating position of mated engagement from a previously proximal position non-mated engagement.

As illustrated at FIGS. 3 and 4, it is preferred that a pair of aligned retention bodies 74 engage a pair of aligned retention elements 76. Such a dual balanced cooperative engagement provides an increased feel to the user of the syringe device. The cooperative engagement of the retention body of the distal piston extension member and the retention element of the barrel extension tip, whether of single or dual engagement fashion, may be either a releasable interference fit or a snap lock fit.

In addition to anti-reflux mechanical structure disposed distal of the stopper 54, 62, the syringe device of the present invention may be further optionally provided with structure proximal of the stopper to inhibit retraction of the stopper from its maximum distally extended position and to prevent withdrawal or release of an elastomeric stopper from any compressed state of the same.

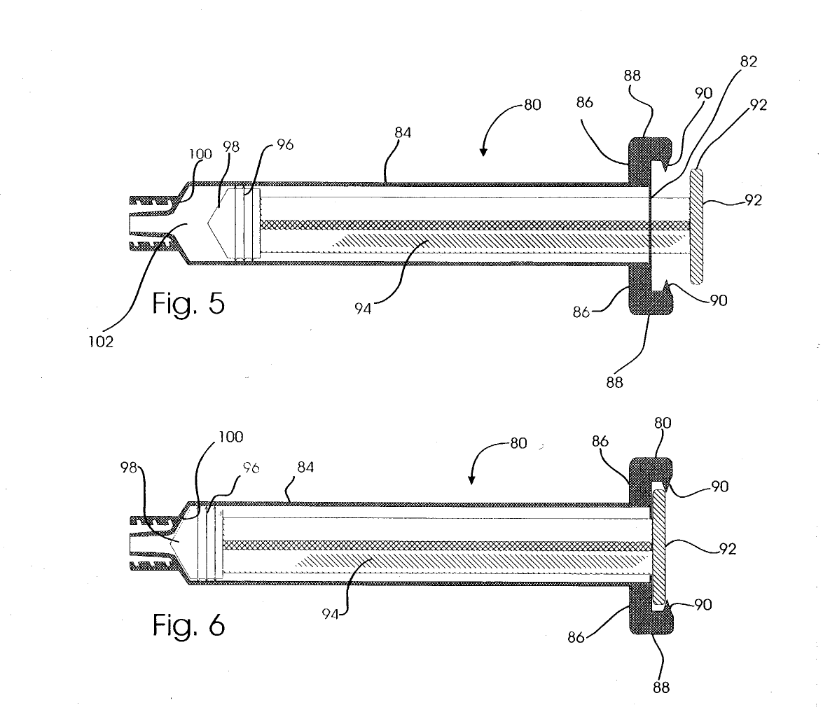

For example, in FIGS. 5 and 6 there is illustrated an alternative embodiment of the present invention as syringe device 80 having the open proximal end 82 of the syringe barrel 84 that terminates into a flange 86. In prior art syringes a syringe barrel flange often serves to limit the distal movement of a piston plunger stem handle such that when the handle contacts the flange, due to the predetermined selected length of the plunger stem and associated piston, the distal movement of a distal seating face of a piston elastomeric stopper can be controlled to be nearly flush with or flush with the interior facing surface of the syringe barrel to avoid or mininize undue compression of the elastomeric stopper. However, such structure relies upon the syringe device user to maintain digital pressure upon the handle and does not mechanically safeguard against release of the handle and an attendant release or withdrawal of the elastomeric stopper from any compressed state. As illustrated at FIGS. 5 and 6, it is preferable that the syringe barrel flange 86 include at least one proximally extending branch 88 having an inwardly extending detent 90 capable of capturing the handle 50 of piston plunger assembly 94 in an affirmative manner which structurally prevents its release thereby preventing any release or decompression of a compressed state of the stopper 96 at its maximum distally extended position. At FIGS. 5 and 6, syringe device 80 has a syringe barrel flange 86 that terminates into a pair of proximally extending branches 88, each of which terminates into inwardly extending detents 90, both capable of capturing the handle 92 when advanced to its maximum distal position. Such capture, illustrated at FIG. 6, affirmatively and structurally prevents an unintended release or retraction of handle 92. Since the total length of the handle 92, the piston plunger assembly 94, and stopper 96 as a measured unit relative to the syringe device barrel flange 86 can be predetermined so as to control, within manufacturing tolerances, the maximum distal movement of the distal seating face 98 of elastomeric stopper 98 and hence its intended degree of “flushness” against distal interior facing surface 100 of syringe barrel chamber 102 to avoid or mininize undue compression of the elastomeric stopper 96, the capturing of the handle maintains the predetermined control and avoids decompression causing reflux.

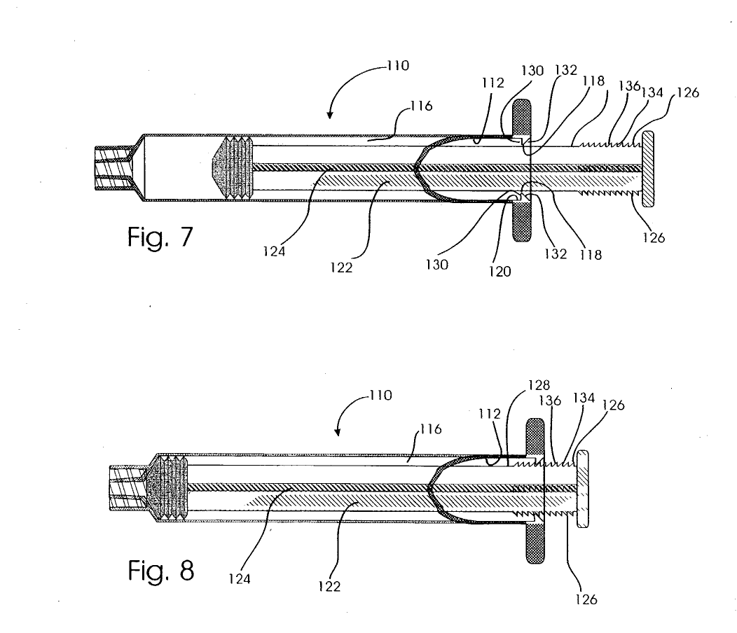

FIGS. 7 and 8 illustrate an alternative embodiment of the present invention as syringe device 110. Syringe device 110 likewise incorporates means proximal of a piston stopper for inhibiting retraction of a distally extended piston stopper. In this regard, the internal surface 112 of the syringe barrel 114 defining the syringe chamber 116 includes one or more inwardly extending detents 118 at a proximal portion 120 thereof and the plunger stem 122 operating the piston plunger assembly 124 includes one or more fins 126 extending from the generally X-shaped in cross section raised outer surfaces 128 of the piston plunger assembly 124. When properly aligned as discussed below, the inwardly extending detents 118 allow distal passage of the fins 126 over the detents while hindering or preventing proximal retraction of the fins over the detents. In this regard, the inwardly extending detents 118 may comprise a distally facing edge 130 that terminates into proximally sloped surface 132 cooperative with a fin 126 comprising a proximally facing edge 134 that terminates into a distally sloped surface 136. The engagement of the respective sloped surfaces of the detents 118 and the fins 126 allow linear passage of the fins while the blockage of their respective facing edges of the detents 118 and the fins 126 prevents linear retraction of the fins. Alignment of the fins relative to the detents can be readily accomplished by a user since the piston plunger assembly 124 is longitudinally and rotationally movable in the syringe barrel 120 by respective longitudinal and rotational movement of its handle. A rotational movement of piston plunger assembly 124 wherein the fins 126 of plunger stem 122 are not aligned with the inwardly extending detents 118 defines a first position that allows unrestricted longitudinal passage of the fins 126 relative to the inwardly extending detents 118. A rotational movement of the piston plunger assembly handle to align the fins with the inwardly extending detents defines a second position wherein the respective sloped surfaces of the detents 118 and the fins 126 allow distal linear passage of the fins over the detents but the respective facing edges of the detents 118 and the fins 126 hinders or prevents proximal retraction of the fins.

Although the syringe device 80 of FIGS. 5 and 6 and the syringe device 110 of FIGS. 7 and 8 each have structural means proximal of the stopper for limiting the distal movement of the stopper and defining a predetermined maximum distal position thereof, there is a significant difference between the two embodiment devices. In the syringe device 80, the total length of the handle 92, the piston plunger assembly 94, and the stopper 96, as measured as a unit, can be captured in the means proximal of the stopper for limiting the distal movement of the stopper and defining a predetermined maximum distal position thereof, namely the inwardly extending detent 90 of the proximally extending branches 88. However, the syringe device 110 is illustrated at FIGS. 7 and 8 with a total length of its handle, piston plunger assembly, and stopper, as measured as a unit, being in excess of the syringe barrel 114. Thus, as observed at FIG. 8, syringe device 110 may allow for a seating of the distal seating face of its elastomeric stopper at a point of “flushness” against the distal interior facing surface of the syringe barrel chamber that still exposes a portion of the plurality of the fins 126 to be proximal of and outside of the inwardly extending detent 118. This “excess” allows for a forced over-compression of the elastomeric stopper against the distal interior facing surface of the syringe barrel chamber while the engagement of fin 126 with inwardly extending detent 118 prevents fluid reflux from a de-compression of the overly compressed elastomeric stopper.

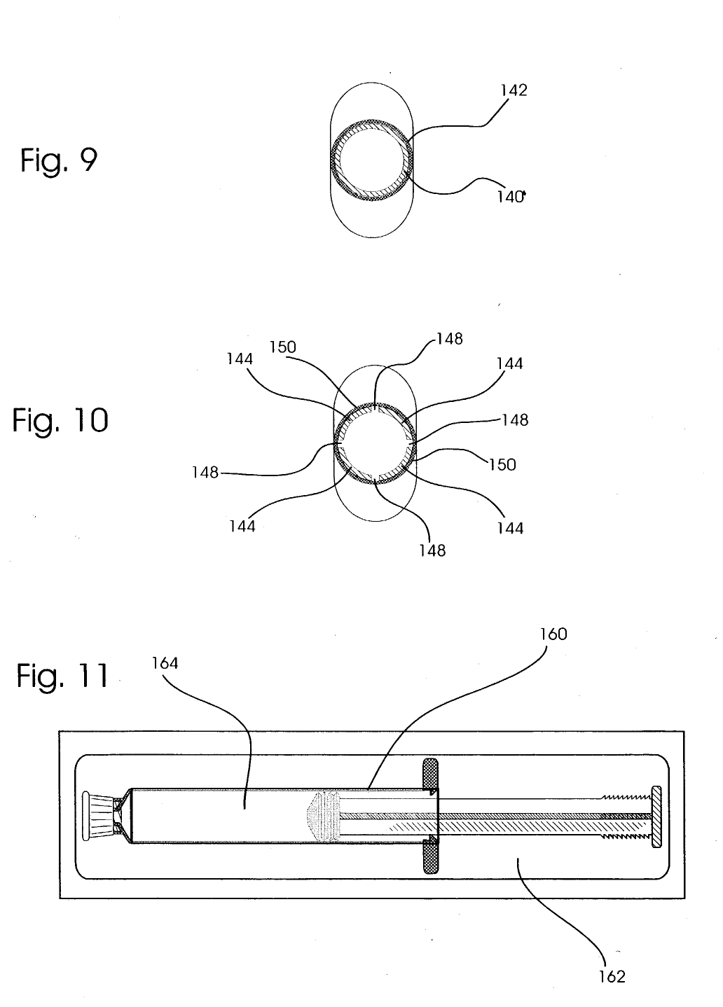

FIGS. 9 and 10 illustrate alternative embodiments of the syringe barrel inwardly extending detent 118. The end view of FIG. 9, illustrates that the inwardly extending detent may take the form of a singular annular ring 140 preferably located at a proximal end portion of the syringe barrel 142. The singular annular ring serves as a constant impediment to proximal retraction of the plunger stem fins after allowing their distal passage. Regardless of the rotational movement of the piston plunger assembly 124 or the radial orientation of the plunger stem fins 126 relative to their linear path along the longitudinal axis of the syringe barrel 142, the fins will encounter the singular annular ring 140.

In contradistinction, at FIG. 10, the inwardly extending detent may take the form of a fragmented annular ring 144, preferably located at a proximal end portion of the syringe barrel 146, that defines fin passage channels 148. Unlike the singular annular ring 140 of FIG. 9, the fragmented annular ring 144 illustrated at FIG. 10 allows for the plunger stem fins 124 to be rotational orientated to the first and second positions previously discussed. In particular, at FIG. 10 there are four fin passage channels defined by the fragmentary nature of the fragmented annular ring 144, the same being cooperatively capable of providing unobstructed longitudinal passage to the plurality fins 126 located at the generally X-shaped in cross section raised outer surfaces 128 of the plunger stem 122 illustrated at FIGS. 7 and 8. The plurality of fins 126 are axially aligned both longitudinally upon the raised outer surfaces 128 of the plunger stem 122 and widthwise of the raised outer surfaces 128 of the plunger stem 122. Only when the piston plunger assembly 124 is rotated such that fins are radially disposed parallel to fin passage channels 148 is their longitudinal distal and proximal passage unobstructed. Other rotational positions of the piston plunger assembly 124 establishing a non-parallel radial orientation of the fins 126 relative to the fin passage channels 148 will have the fins encounter the fragmented annular ring 144.

FIG. 11 illustrates a pre-filled assembled syringe device 160 of the present invention that is provided in a tearable or frangible package 162 that provides a tamper evident barrier and a sterile barrier surrounding the syringe device. Packaging that provides substantial resistance to the passage of microorganisms can be made of many known materials such as paper, coated paper, plastic film, foil, non-woven materials and combinations thereof can be used as package 162 for such purposes. Tearing of the package 162 may be evidence that the syringe has been used or tampered with and the sterile barrier compromised. In making the packaged syringe assembly, syringe device can be sterilized after filling with pre-fill solution, for example saline flush solution or heparin lock flush solution, and placed in the package and sterilized a second time.

The syringe device of the present invention can be used to flush catheters having I.V. sites in a manner similar to prior art syringes. When a syringe barrel assembly and a piston plunger assembly are assembled to form the syringe device, the elongated plunger stem extends proximally from the stopper through the open proximal end of the syringe barrel. After removal of the sealing luer cap, the user's digital pressure upon handle drives the plunger stem distally directing its piston and stopper distally to discharge the injectable fluid in the syringe barrel chamber through barrel extension tip into and through the catheter.

The features of the present invention heretofore discussed in reference to FIGS. 1 through 10 provide for multiple controls for the elastomeric stopper of a syringe barrel at a point distal and/or proximal of the stopper to avoid excessive compression of the stopper that, upon release of the plunger stem may draw blood into a catheter during the flush procedure. Such features may be mixed and combined in various embodiments of the present Anti-Reflux Syringe invention.

The syringe device of the present invention advantageously both limits the distal movement of the plunger stem piston stopper to a predetermined maximum distal position avoiding excessive compression of the elastomeric stopper and inhibits retraction of the stopper from a distally extended position of the same by structure distal and/or proximal of the stopper. A maximum distal position of the plunger stem piston stopper can be set by the distal piston extension member engaging the syringe barrel extension tip, by the preferred structure of the retention body at the outer periphery of the distal piston extension member seating in interference fit or lock fashion within the cooperative retention element at the interior surface of the syringe barrel extension tip, and/or by a handle of the piston stem contacting a flange of the open proximal end of the syringe barrel. Structure inhibiting retraction of the stopper from a distally extended position of the same may be distal of the stopper, as is the case when the retention body at the outer periphery of the distal piston extension member seats in interference fit or lock fashion within the cooperative retention element at the interior surface of the syringe barrel extension tip, and/or may be proximal of the stopper as is the case when the detent of the proximally extending branch of the proximal open end syringe barrel flange captures the piston stem handle or when the inwardly extending detent internal of the syringe barrel hinders or prevents proximal retraction of the fins upon the piston stem.

The syringe device of the present invention may be pre-assembled or may comprise separate syringe barrel assembly and plunger piston assembly component parts for ready assembly if purposes such as conserving space or reducing packaging material are desirable. Likewise, the piston and its distal piston extension member may be assembled to the elastomeric stopper at the time of use by simply screwing the piston into the stopper so as to distally expose the distal piston extension member through the bore of the stopper. The threaded arrangement described hereinabove is considered exemplary of these many possibilities. It is also within the purview of the present invention to include a one-piece piston, stopper, and distal piston extension member assembly wherein the foregoing components stopper made of the same material. Still further, there are many ways to connect the stopper to a piston including snap-fit structure, adhesives, welding and two-shot molding where a stopper of one material is molded with a plunger stem of another material.

From the foregoing description, it will be apparent that the Anti-Reflux Syringe of the present invention has a number of advantages, some of which have been described above and others of which are inherent in the invention. Also it will be understood that modifications can be made to the Anti-Reflux Syringe described above without departing from the teachings of the present invention. Accordingly, the scope of the invention is only to be limited as necessitated by the accompanying claims and their equivalents.

I claim:

1. A syringe device comprising:
a syringe barrel having an elongated body with an external surface and an internal surface defining a chamber for retaining fluid, an open proximal end, a distal portion, and a barrel extension tip extending from said distal portion, said barrel extension tip having a tip passageway therethrough in fluid communication with said chamber;
a piston disposed in said syringe barrel for controlling the flow of fluid within said elongated body, said piston having a distal piston extension member capable of engaging said barrel extension tip;
a plunger stem having a distal end operatively connected to said piston and a proximal end terminating into a handle, said handle being dispose to extend proximally from said open proximal end of said syringe barrel, said plunger stem being movable with said piston; and
a stopper integral with or releaseably mounted upon said piston in fluid tight communication inside said chamber, said stopper having a bore at a distal portion thereof through which said distal piston extension member extends.

2. The syringe device of claim 1 wherein said distal piston extension member further includes a retention body at a portion of its outer periphery and said barrel extension tip further includes a retention element at an inner surface thereof, said retention body of said distal piston extension member being dimension to cooperatively engage said retention element of said barrel extension tip at a position distal of said stopper, such cooperative engagement limiting the distal movement of said stopper and defining a predetermined maximum distal position thereof, and forming a barrier to proximal withdrawal of the stopper.

3. The syringe device of claim 2 wherein said retention body of said distal piston extension member comprises a male protrusion and said retention element of said barrel extension tip comprises a female reception area.

4. The syringe device of claim 2 wherein said cooperative engagement of said retention body of said distal piston extension member and said retention element of said barrel extension tip comprises a releasable interference fit.

5. The syringe device of claim 2 wherein said cooperative engagement of said retention body of said distal piston extension member and said retention element of said barrel extension tip comprises a snap lock fit.

6. The syringe device of claim 1 wherein said barrel extension tip and said distal piston extension member are each frusto-conically shaped.

7. The syringe device of claim 1 wherein said barrel extension tip is externally threaded to engage the internal threads of a luer cap.

8. The syringe device of claim 1 wherein said stopper is made of an elastomeric material selected from the group of natural rubber, synthetic rubber, thermoplastic elastomers, or combinations thereof.

9. The syringe device of claim 1 wherein said piston is externally threaded to releaseably engage the internal treads of said stopper.

10. The syringe device of claim 1 wherein said plunger stem is made of a rigid thermal plastic material.

11. The syringe device of claim 1 wherein said plunger stem is substantially X-shaped in cross-section.

12. The syringe device of claim 1 wherein said open proximal end of said syringe barrel terminates into a flange, said flange limiting the distal movement of said piston when contacted by said handle.

13. The syringe device of claim 12 wherein said flange further includes a proximally extending branch having an inwardly extending detent capable of capturing said handle.

14. The syringe device of claim 1 wherein said internal surface of said elongated body further includes an inwardly extending detent and said plunger stem includes a fin extending from its outer surface, said detent being capable of allowing distal passage of said fin and either hindering or preventing proximal retraction thereof.

15. The syringe device of claim 14 wherein said detent comprises a distally facing edge terminating into a proximally sloped surface and said fin comprises a proximally facing edge terminating into a distally sloped surface.

16. The syringe device of claim 14 wherein said detent comprises an annular ring.

17. The syringe device of claim 14 wherein said detent comprises a fragmented annular ring defining channels capable of allowing unobstructed distal and proximal longitudinal passage of said fin.

18. The syringe device of claim 14 wherein said internal surface of said elongated body includes a plurality of inwardly extending detents.

19. The syringe device of claim 14 wherein said plunger stem includes a plurality of axially aligned fins extending from its outer surface.

20. The syringe device of claim 19 wherein said axial alignment of said plurality of fins is longitudinal upon said plunger stem.

21. The syringe device of claim 19 wherein said axial alignment of said plurality of fins is widthwise of said plunger stem.

22. The syringe device of claim 1 wherein said syringe barrel further includes communicative indicia.

23. The syringe device of claim 1 wherein said syringe device is contained in a package that provides a sterile barrier surrounding the syringe device.

24. The syringe device of claim 1 wherein said syringe device is contained in a package that provides a tamper evident barrier surrounding the syringe device.

25. The syringe device of claim 7 wherein said chamber is pre-filled with a flush solution.

26. The syringe device of claim 25 wherein the flush fluid solution is selected from the group consisting of saline flush solution and heparin lock flush solution.

27. A syringe device comprising:
a syringe barrel having an elongated body with an external surface and an internal surface defining a chamber for retaining fluid, an open proximal end, a distal portion, and a barrel extension tip extending from said distal portion, said barrel extension tip having a tip passageway therethrough in fluid communication with said chamber;
a piston disposed in said syringe barrel for controlling the flow of fluid within said elongated body,
a plunger stem having a distal end operatively connected to said piston and a proximal end terminating into a handle, said handle being disposed to extend proximally from said open proximal end of said syringe barrel, said plunger stem being movable with said piston;
a stopper integral with or releaseably mounted upon said piston in fluid tight communication inside said chamber, and
means proximal of said stopper for inhibiting proximal retraction of said stopper from a distally extended position of the same.

28. The syringe device of claim 27 further including means proximal of said stopper for limiting the distal movement of said stopper and defining a predetermined maximum distal position thereof.

29. The syringe device of claim 28 wherein said means proximal of said stopper for limiting the distal movement of said stopper and defining a predetermined maximum distal position thereof comprises said open proximal end of said syringe barrel terminating into a flange, said flange limiting the distal movement of said piston when contacted by said handle.

30. The syringe device of claim 29 wherein said means proximal of said stopper for inhibiting proximal retraction of said stopper from a distally extended position of the same comprises said open proximal end of said syringe barrel terminating into a flange, said flange further including a proximally extending branch having an inwardly extending detent capable of capturing said handle adjacent said flange.

31. The syringe device of claim 27 said means proximal of said stopper for inhibiting proximal retraction of said stopper from a distally extended position of the same comprises said internal surface of said elongated body further including an inwardly extending detent and said plunger stem including a fin extending from its outer surface, said detent being capable of allowing distal passage of said fin and either hindering or preventing proximal retraction thereof.

32. The syringe device of claim 31 wherein said plunger stem is longitudinally and rotationally movable in said syringe barrel by respective longitudinal and rotational movement of said handle, said rotational movement allowing unrestricted longitudinal passage of said fin of said plunger stem relative to said inwardly extending detent of said elongated body when said fin is not aligned with said inwardly extending detent.

33. The syringe device of claim 32 wherein said unrestricted longitudinal passage of said fin of said plunger stem relative to said inwardly extending detent of said elongated body defines a first rotational position of said fin relative said inwardly extending detent, and further rotational movement to align said fin with said inwardly extending detent defines a second rotational position of said fin relative said inwardly extending detent, said second rotational position either hindering or preventing proximal retraction of said stopper from a distally extended position of the same.

34. The syringe device of claim 31 wherein said detent comprises a distally facing edge terminating into a proximally sloped surface and said fin comprises a proximally facing edge terminating into a distally sloped surface.

35. The syringe device of claim 31 wherein said detent comprises an annular ring.

36. The syringe device of claim 31 wherein said detent comprises a fragmented annular ring defining channels capable of allowing unobstructed distal and proximal passage of said fin.

37. The syringe device of claim 31 wherein said internal surface of said elongated body includes a plurality of inwardly extending detents.

38. The syringe device of claim 31 wherein said plunger stem includes a plurality of axially aligned fins extending from its outer surface.

39. The syringe device of claim 38 wherein said axial alignment of said plurality of fins is longitudinal upon said plunger stem.

40. The syringe device of claim 38 wherein said axial alignment of said plurality of fins is widthwise of said plunger stem.

41. The syringe device of claim 27 wherein said syringe barrel further includes communicative indicia.

42. The syringe device of claim 27 wherein said syringe device is contained in a package that provides a sterile barrier surrounding the syringe device.

43. The syringe device of claim 27 wherein said syringe device is contained in a package that provides a tamper evident barrier surrounding the syringe device.

44. The syringe device of claim 27 wherein said chamber is pre-filled with a flush solution.

45. The syringe device of claim 44 wherein the flush fluid solution is selected from the group consisting of saline flush solution and heparin lock flush solution.
